(19) United States
(12) Patent Application Publication
(10) Pub. No.: US 2007/0255186 A1
(43) Pub. Date: Nov. 1, 2007

(54) Products and methods for motor performance improvement in patients with neurodegenerative disease

(76) Inventor: Stephen Grill, Elkridge, MD (US)

Correspondence Address: CONWELL LLC, 2138 PRIEST BRIDGE COURT, SUITE 4, CROFTON, MD, 21114, US

(21) Appl. No.: 11/742,840

(22) Filed: May 1, 2007

Related U.S. Application Data
(60) Provisional application No. 60/796,582, filed on May 1, 2006.

(51) Int. Cl. A61B 5/103

(52) U.S. Cl. 600/595

(57) ABSTRACT

The present invention is drawn to products and methods to improve the gait performance of subjects with neurodegenerative disease before and after feedback-enhanced training. The present invention has the goal of producing larger stride length in patients with neurodegenerative disease. The method includes patients walking on a treadmill while a bright line is shown at increasing distances from their center of gravity, and encouraging them to take larger steps. The present invention also includes a walking assistance appliance, including, a walker, cane, and so forth.

### BACKGROUND

1. Field of the Invention

This invention relates to products and methods for improving motor performance in patients with neurodegenerative conditions.

2. Background of the Invention

Neurodegenerative diseases, such as Guillain-Barre syndrome, Huntington's disease and amyotrophic lateral sclerosis (ALS), multiple sclerosis (MS) and Parkinson's Disease (PD) and injury caused by stoke, atherosclerosis, traumatic injury from accident, and the like, afflict patients with a reduced ability of movement. Parkinson's Disease is a progressive neurodegenerative disease that causes affected individuals to move slowly and make small movements. Patients with Parkinson's display tremor, rigidity, bradykinesia and postural instability. Without the use of exteroceptive (visual or auditory) input, patients make hypometric movements (Flowers, Brain, 99: 269-310, 1976, Klockgether & Dichgans, Mov. Disord., 9: 48-56, 1987) but motor performance improves with auditory or visual cues (Georgiou et al., Brain, 116: 1575-78, 1993).

The gait in PD patients may be described as shuffling, with short hesitant steps which are sometimes quick (festinating). They have difficulty initiating their gait and walk stiffly with limited arm swing. The postural instability is usually a relatively late symptom of the disease and one that is not amenable to current medical or surgical therapy (Koller et al, Clin. Neuropharmacol., 12: 98-105, 1989), although some improvement in balance has been reported with bilateral subthalamic stimulators (Bejjani et al., Jour. Neurol. Neurosurg. Psych. 68(5): 595-600, 2000). Patients experiencing postural instability are at increased risk of falls resulting in traumatic injuries and are usually dependent on the use of assistive devices such as walkers. In one survey, PD patients had a yearly incidence of broken bones of 35.6% of which ⅓ were hip fractures (Pressley et al., Neurology, 60(1): 87-93, 2003). Following hip fractures, the gait worsens and 21.9% may be filly unable to walk (Gialanella, Minerva Med., 92(3): 11-6, 2001). In addition, freezing and gait hesitation usually occur relatively late in the disease and can be quite debilitating even when the other symptoms of the disease are well-treated medically.

Although patients are routinely sent for physical therapy to address their gait problems, the efficacy is not well documented. Furthermore, the methods used vary from center to center and have not been subjected to rigorous scientific investigation. Weight-supported treadmill training, a technique in which the patient walks on a treadmill with partial body weight support through an overhead hairless as well as a pelvic belt has been found to improve gait stride length and speed in persons with strokes (Miyai et al., Arch. Phys. Med. Rehabil 81: 849-52, 2000) and these benefits may be long-lasting (Miyai et al., Arch. Phys. Med. Rehabil., 83: 1370-3, 2002). The mechanism for the improvement is unknown. A portion of the improvement with treadmill training in PD may be due to aerobic conditioning since that seems to be a factor when applied to stroke patients (Macko et al. Stroke, 28: 326-330, 1997; Macko et al., Arch. Phys. Med. Rehab.) 82(7): 879-884, 2001). In this training in PD patients, the weight support may be a factor (Visintin et al., Stroke, 29: 1122-28, 1998) in the improvement aside from any improvements resulting from more efficient energy expenditure. Frenkel-Toledo et al. (Movement Disorders 20(9): 1109-1114, 2005) suggest that the treadmill itself may be acting as an external cue to enhance the rhythmicity of the gait of the PD patient, but did not demonstrate any stride length improvements.

It is a common observation that patients with PD may undergo severe “freezing” when attempting to go through door ways but may have little trouble going up stairs or when there is a repeated pattern on the floor. These visual stimuli can have large effects on a patient's gait. There is a commercially-available cane (STEPOVER WAND®) which employs a red wire as a visual stimulus, a visual aid for patients experiencing freezing. With this device, patients are explicitly using visual input to help improve the magnitude of their steps. It is well accepted that visual stimuli may improve gait by alleviating freezing, and in fact, there are improvements in gait in PD patients with visual and auditory cueing (Suteerawattananon et al., J. Neurol Sci., 219: 63-69, 2004). However, the improvements with auditory cueing (using a metronome) were in cadence rather than stride length (Suteerawattananon et al., Ibid, 2004).

The use of the term “gait improvement,” as used herein, means the alleviation of freezing and hesitation.

The short steps that PD patients take are one form of the hypometria they experience and this is also present during movements of the upper extremities as well. With visual feedback, patients are, however, able to make larger limb movements and the deficit may be due to a sensory-motor mismatch (Demerci et al. Ann. Neurol., 41: 781-788, 1997). That is, the kinesthetic signal may be “felt” as indicating the person has made an adequately-sized movement even though they did not. They perceive distances to be shorter than control subjects when they use kinesthesia rather than vision. PD patients often feel they are speaking at a normal volume even though they may be severely hypophonic (Marsden, Neurology, 32: 514-539, 1982), but when they are coerced into speaking louder they feel as if they are shouting (Ramig et al, J. Med. Speech Lang. Pathol., 2: 191-209, 1994). Apparently during both limb movements and speech, PD patients have a feeling of performing well and do not attempt to make collections because they do not feel any discrepancy between their motor intention and performance (motor output) as long as there is no exteroceptive feedback. Based on these ideas, improvements in speech have been achieved (the Lee Silverman Voice Treatment program). The focus is on producing a louder volume and this results in improvements in articulation as well (Ramig, Intelligibility in Speech Disorders: Theory, Measurement and Management, John Benjamins Pub. Co., R. Kent, ed., Amsterdam, 1992) even though articulation is not stressed during the therapy. These ideas have not been applied to physical therapies for gait.

U.S. Pat. No. 6,704,603 B1 and divisional U.S. application 2004/0133249 A1 describe a method of adaptive stimulation and an adaptive stimulator product. The '603 patent describes a control unit and method to aid in the relief of symptoms of Parkinson's disease. The device disclosed electrically stimulates a person's muscles at a set rhythm to stimulate better movement or uses a signal to tell the patient when to take a step. Stride lengthening is not disclosed.

Since neurodegenerative disease patients have a greatly increased risk of suffering traumatic injuries as a result of postural instability, intervention with physical therapies and the use of assistive devices, such as canes and walkers, may be helpful in preventing these falls. Various experimental therapy methods including Body Weight Supported Treadmill Training (Miyai et al., Ibid, 2000, 2002), and visual and auditory cueing (Suteerawattananon et al, Ibid, 2004) have demonstrated some improvement in gait parameters. However, there are no standardized physical therapeutic modalities which lead to improvement in gait or postural stability. There is a need in the art for improving the gait stride length and speed of a patient, as well as improving postural stability in patients with neurodegenerative disease.

### SUMMARY OF THE INVENTION

The present invention is drawn to products and methods to improve gait performance of subjects before and after feedback-enhanced training.

### BRIEF DESCRIPTION OF THE FIGURES

FIG. 1 depicts a feedback-enhanced treadmill device as viewed from the top.

FIG. 2A shows a walker device as viewed from the side. FIG. 2B shows a full view of a walker with a gait training device installed.

FIG. 3 displays improvement in gait in a patient with idiopathic Parkinson's disease during feedback-enhanced treadmill training.

### DETAILED DESCRIPTION OF THE INVENTION

The present invention is drawn to the use of feedback methods during treadmill walking with the goal of producing larger stride length in patients with neurodegenerative disease. The invention includes patients walking on a treadmill while a bright line is shown, via an LED, at increasing distances from their center of gravity, and encouraging them to take larger steps. With each step an auditory tone will be produced if their step size reaches the target stride length. The therapist continuously encourages and promotes the larger steps during the training. By employing the visual and auditory feedback of the invention, patients will be able to increase their stride length while walking on the treadmill. Patients experience appropriate proprioceptive feedback from their leg muscles and thus correct the sensory-motor mismatch in a similar way as it seems to do in the Lee Silverman Voice Treatment program (Ramig, Intelligibility in Speech Disorders: Theory, Measurement and Management, John Benjamins Pub. Co., R. Kent, ed., Amsterdam, 1992).

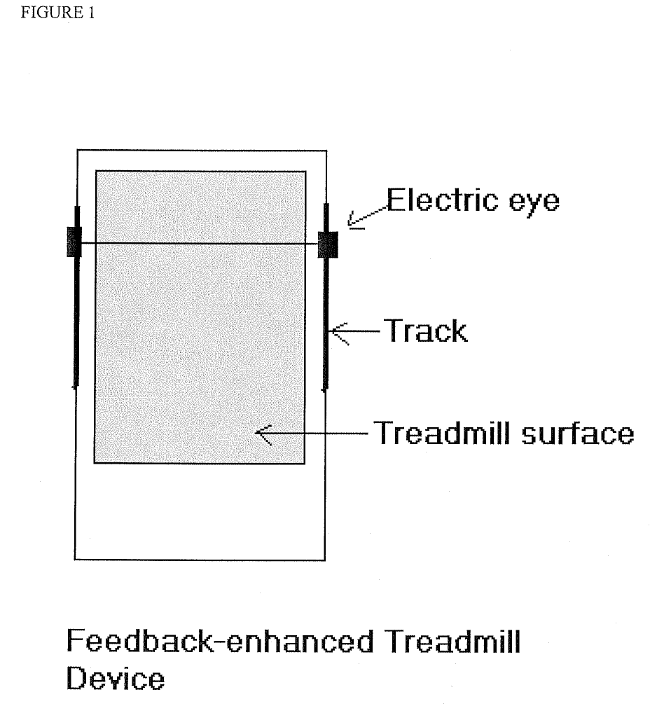

FIG. 1 displays a diagram of one embodiment of the invention comprising a treadmill surface with an electric eye adjustably affixed to a track thereto. A laser source may be affixed to the apparatus so that a beam can be adjustably projected onto the treadmill surface as determined by a practitioner. Said adjustably projectable laser beam, or other visual means of projecting a visual cue to the patient, allows a lengthening of the distance from the patient to affect a longer stride length. Such visual cues includes but are not limited to laser light, phosphorescent strips, backlit light sources, which require a translucent or transparent treadmill surface, wires of clearly distinguishable textures or colors, projected lights or images, and so forth.

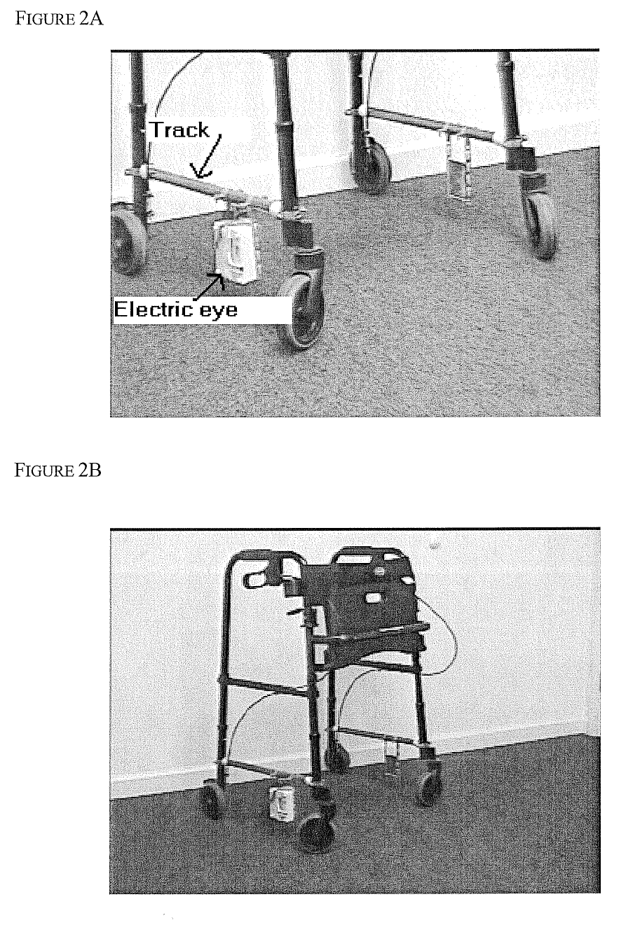

FIG. 2A displays a diagram of a walker device comprising a treadmill surface with an electric eye adjustably affixed to a track thereto. A laser source may be affixed to the apparatus so that a beam can be adjustably projected onto the walking surface as determined by a practitioner. Said adjustably projectable laser beam, or other visual means of projecting a visual cue to the patient, allows a lengthening of the distance from the patient to affect a longer stride length. Such visual cues includes but are not limited to laser light, phosphorescent strips, backlit light sources, which require a translucent or transparent walking surface, wires of clearly distinguishable textures or colors, projected lights or images, and so forth. FIG. 2B shows the entire walker device with the attached apparatus. Such a walking device is shown as one embodiment of the invention and can also include, without being limited to other types of “walker” devices, wheeled rollators, canes, chair/canes devices, crutches, 0, 2, 3, 4 or more wheeled walker or walking-assistance devices, and the like.

The present invention includes a method for the evaluation of stride length and gait speed. Patients undergo baseline gait evaluation 1 week prior to the beginning of the treadmill training as well as at the beginning of each training session.

The Unified Parkinson's Disease Rating Scale (UPDRS) can be performed at each visit. The “Functional Reach” is a simple method to assess postural stability and, has been shown to predict falls in elderly men (Duncan et al, J. Gerontol., 47(3): M93-98, 1992) and has been applied in a limited fashion in patients with Parkinson's disease (Grill, Maryland Med J, July/August: 179-181, 1999). It involves having a patient stand upright with the right arm outstretched at a 90 degree angle to the body reaching across a tape measure fixed to a wall. The patient is instructed to reach along the tape measure as far as possible until they lose their balance, with the therapist present to prevent actual falls. The further the patient is able to reach, the better the balance. Studies indicate that postural instability (and greater risk for falls) is present when the patient can reach less than 20 cm.

Additionally, the invention includes a method of treadmill training. Patients walk on the treadmill for 3 sessions of 10 minutes each with a break of 10 minutes between sessions. Patients should be secured into a frame, which completely encircles the user to provide protection from falling in any direction, such as the TREADABOUT harness (innovative Health Solutions), or the like. This also ensures that the patient does not advance or regress on the treadmill and instead that they will remain in the center of it. The target stride length (SLₜ) at the beginning of each session will is defined as SLₜ=SLᵢ+SLᵢ*0.2, where SLᵢ is the mean stride length determined on the gait mat on the same day prior to initiation of the treadmill training. This represents a 20% increase in stride length compared to baseline. A laser line can serve as a visual target that is projected onto the treadmill surface at a set distance from the center of a patient support harness. The initial speed of the treadmill should be at the same speed as the patient was able to walk at the beginning of the session on the gait mat. In line with the laser line can be an electric eye, such as produced by Blue Point Engineering and the like, such that each time the patient makes a step with the target stride length, the eye is interrupted. The signal from the eye can be input into a computer programmed to store the number of times the target is achieved. One embodiment can include the use of a commercially-available software program such as that produced by Asyst Technologies, Inc. (Fremont, Calif.), or any software or any high level language to perform A/D and digital input/output. With each step that achieves the target stride length, a tone can be immediately produced giving the patients explicit feedback as to whether they in fact achieved the target stride length. Without being limited, the feedback can include tones, visual stimuli, such as lights, and/or symbolic data, such as a gauge, LED display or paper printout and/or the stimulus could be tactile in nature. Thus the patient can receive visual, tactile and/or auditory feedback of their performance. A second electric eye can be positioned closer to the patient in a way to capture each step they take regardless of whether they achieve the target stride length. Since gait speed (V)=SL times the number of steps/time, the number of steps/time will decrease although the gait speed may increase. The practitioner can increase the speed of the treadmill as needed to achieve a speed comfortable to the patient. The primary goal of the patient is to increase the stride length. When the patient is producing the target stride length 75% of the time over 50 steps, the stride length will be increased by 10% with the maximum target stride length being 40% of their height (Suteerawattananon et al. Ibid, 2004).

Neurodegenerative conditions that can be treated using the methods and products of the invention include: Idiopathic Parkinson's Disease and other Parkinsonian syndromes, Senile Gait Disorder, Ataxia and other gait disorders.

Patients include human and non-human patients such as farm animals including horses, pets, such as dogs, and research animals such as non-human primates. The methods and products described herein can be used for strength training for healthy individuals in sports training and/or as part of a physical health regimen.

The invention includes a walking device comprising a walking surface, an electric eye adjustably affixed to a sliding track thereto, and a means of projecting a visual cue affixed to the device so that a beam can be adjustably projected onto the walking surface. The invention additionally includes the walking device, wherein said adjustably projectable visual cue is a laser light, and/or wherein the walking device wherein the walking surface is a treadmill. The invention includes the walking device, wherein the walking device is selected from the group consisting of walkers, wheeled rollators, canes, chair/cane devices, and crutches.

A method of the invention includes a method of evaluation of stride length and gait speed comprising providing a walking device comprising a walking surface, an electric eye adjustably affixed to a sliding track thereto, and a means of projecting a visual cue affixed to the device so that a beam can be adjustably projected onto the walking surface;
securing a patient in a patient support;
projecting a visual target onto said walking surface at a set distance from the center of the patient support;
providing an initial speed of said walking surface at the same speed as the patient was able to walk at the initiation of a session;
providing in line with the laser line, an electric eye, such that each time the patient makes a step with the target stride length, the eye is interrupted;
wherein a signal generated from the eye can be input into a computer programmed to store the number of times the target is achieved;
wherein with each step that achieves the target stride length, a tone is produced to give a patient an explicit feedback as to whether the target stride length was achieved; and
comparing the stride length with an initial stride length as measured on an unmoving gait mat which captures stride length, cadence, and velocity of gait.

The method of the invention additionally includes a method of evaluation of stride length and gait speed wherein the feedback is selected from a group including auditory tones, tactile feedback and visual feedback selected from a group consisting of projected light, laser line, projected images, a gauge, LED display and paper printout.

### WORKING EXAMPLES

### Example 1

### Gait Training in PD Patients

Inclusion/Exclusion criteria: Patients with Idiopathic Parkinson's Disease, using UK brain bank criteria (Hughes et al. 1992), Hughes A J, Daniel S E, Kilford L, Lees A J. “Accuracy of clinical diagnosis of idiopathic Parkinson's disease: A clinicopathological study of 100 cases” (J Neurol Neurosurg Psychiatry 1992; 55: 181-184. Hoehn & Yahr) stages II-IV were included as long as they were able to ambulate independently either with or without an assistive device. They had a stride length that is less than 40% of their height (Suteerawattananon et al, Ibid, 2004). Patients with significant cardiovascular disease, with a history of unstable angina, recent (<3 months) myocardial infarction, congestive heart failure or hemodynamically significant valvular disease were excluded and medical clearance from their internist was required for participation. Patients with neuropathy (based on clinical examination of vibratory and position sensation at the feet) were excluded because their impaired proprioception could confound their ability to use proprioceptive information during the treadmill training. Patients with significant dementia as defined by a MMSE<24 were excluded. Patients were required to be on a stable anti-Parkinson's drug regimen and were excluded if they could not remain on their current regimen throughout the duration of the study. We enrolled 3 patients in this study.

The methods of the invention included the effects of three months of feedback-enhanced treadmill training in PD patients on gait. We identified and recruited patients who were ambulatory with or without an assistive device to determine their eligibility for the study. They were screened with medical and neurological examinations, the Unified Parkinson's Disease Rating Scale, Hoehn and Yahr staging, Mini Mental State Examinations, and a gait evaluation as described above. Patients were randomized to receive conventional physical therapy by a licensed physical therapist, or the treadmill training also supervised and administered by a licensed physical therapist. Patients were required to attend 90% of the scheduled treatment sessions. Patients underwent therapy 3 times per week for 12 weeks. Evaluations were performed at baseline, at each visit, and at 4 weeks and 12 weeks after completing the 12 weeks of therapy.

In addition to the subscore of the Activities of Daily Living (ADL) section in which a history of falls is queried, patients will be asked to keep an accurate diary of any falls, including the circumstances of falls, in the preceding week.

### Statistical Analysis

The change in stride length, gait speed, and Functional Reach was evaluated with paired t-tests within subjects in the treadmill-trained group. The t statistic is used to evaluate the difference between the treadmill-trained group and the group undergoing standard physical therapy. These analyses are performed at weeks 4 and 12 as well as at 4 and 12 weeks after finishing training (weeks 16 and 24) in order to assess long term effects. The sample size for each group is calculated to be 11. A 10% variability of stride length with a mean difference of 10% in stride length, pre and post treadmill training, at p<0.05 was noted. We will also compare using a paired t-test the frequency of falls before and after treadmill training in the treadmill-treated group, and the treadmill-treated group to those treated with conventional physical therapy.

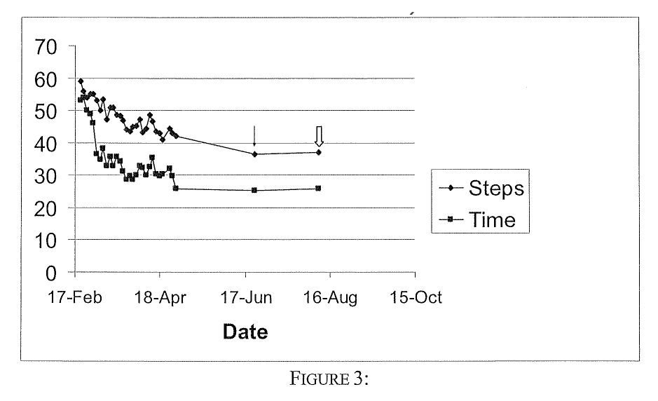

The data of FIG. 3 display striking improvement in a patient with idiopathic Parkinson's disease who completed 12 weeks of feedback-enhanced treadmill training. Note that the patient initially took 60 steps and 54 seconds to walk 12 meters. At the end of the therapy, he was able to walk the 12 meters in 25 seconds taking 42 steps. The improvement persisted 2 months later despite not having continued therapy (thin arrow), and 2 months later during which time he used the modified walker (thick arrow).

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are incorporated by reference in their entirety.

We claim

1. A walking device comprising a walking surface, an electric eye adjustably affixed to a sliding track thereto, and a means of projecting a visual cue affixed to the device so that a beam can be adjustably projected onto the walking surface.

2. The walking device of claim 1 wherein said adjustably projectable visual cue is a laser light.

3. The walking device of claim 2 wherein said walking surface is a treadmill.

4. The walking device of claim 2 wherein said walking device is selected from the group consisting of walkers, wheeled rollators, canes, chair/cane devices, and crutches.

5. A method of evaluation of stride length and gait speed comprising providing a walking device comprising a walking surface, an electric eye adjustably affixed to a sliding track thereto, and a means of projecting a visual cue affixed to the device so that a beam can be adjustably projected onto the walking surface;
securing a patient in a patient support;
projecting a visual target onto said walking surface at a set distance from the center of the patient support;
providing an initial speed of said walking surface at the same speed as the patient was able to walk at the initiation of a session;
providing in line with the laser line, an electric eye, such that each time the patient makes a step with the target stride length, the eye is interrupted;
wherein a signal generated from the eye can be input into a computer programmed to store the number of times the target is achieved;
wherein with each step that achieves the target stride length, a tone is produced to give a patient an explicit feedback as to whether the target stride length was achieved; and
comparing the stride length with an initial stride length as measured on all unmoving gait mat which captures stride length, cadence, and velocity of gait.

6. The method of claim 5 wherein said feedback is selected from a group consisting of auditory tones, tactile feedback and visual feedback selected from a group consisting of projected light, laser line, projected images, a gauge, LED display and paper printout.
